(19) United States
(12) Patent Application Publication
(10) Pub. No.: US 2006/0223780 A1
(43) Pub. Date: Oct. 5, 2006

(54) Method for administration of capecitabine

(76) Inventors: Ute Dugan, New York, NY (US); Larry Norton, New York, NY (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/388,881

(22) Filed: Mar. 24, 2006

Related U.S. Application Data
(60) Provisional application No. 60/667,509, filed on Apr. 1, 2005.

(51) Int. Cl. A61K 31/7072

(52) U.S. Cl. 514/49

(57) ABSTRACT

There is provided a new dosage regimen for capecitabine which maximizes anti-tumor activity while maintaining acceptable toxicity levels.

### PRIORITY TO RELATED APPLICATIONS

This application claims the benefit of U.S. Provisional Application No. 60/667,509, filed Apr. 1, 2005, which is hereby incorporated by reference in its entirety.

### FIELD OF THE INVENTION

The present invention is directed to improved methods of administration of capecitabine in the treatment of cancer. In particular, the invention is directed to improved methods of administration of capecitabine that provide desirable antineoplastic effects with a tolerable level toxicity. The methods of the invention are characterized by administering less frequent doses comprising relatively high concentrations of capecitabine. This protocol is both safer and more efficacious than administering frequent doses of lower concentrations.

### BACKGROUND OF THE INVENTION

Capecitabine is a fluoropyrimidine carbamate with antineoplastic activity. It is an orally administered systemic prodrug of 5′-doexy-5-fluorouridine (5′-DFUR) which is converted to 5-fluorouracil intracellularly, an antineoplastic agent. Capecitabine is marketed in the United States by Roche Laboratories under the brand name Xeloda®. The chemical name for capecitabine is 5′-deoxy-5-fluoro-N-[(pentyloxy)-carbonyl]-cytidine and has the following structural formula:

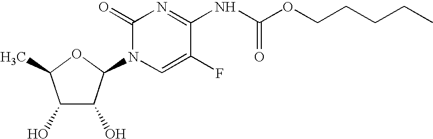

Capecitabine is covered in US patents, including U.S. Pat. Nos. 4,966,891 and 5,472,949. Improved methods for the manufacture of capecitabine are also taught by U.S. Pat. Nos. 5,453,497 and 5,476,932, and application U.S. Ser. No. 60/532,266, filed Dec. 22, 2003. To the extent necessary, any and all of the foregoing patents and applications are herein incorporated by reference.

Capecitabine is currently approved for the treatment of colon and breast cancer. The currently approved/recommended dose of capecitabine in those indications is 1250 mg/m² administered orally twice daily (equivalent to 2500 mg/m² total daily dose) for 14 days followed by a 7 day rest period given as 3-week cycles, for as long as needed. See approved package insert. Typically the mean duration of treatment is 3 to 6 three-week cycles.

A draw back with current capecitabine therapy is the advent of hand-foot syndrome (palmar-plantar erythrodysesthesia or chemotherapy-induced acara erythema). The median time to onset of this side effect is about 79 days into therapy with the approved dose and schedule of capecitabine. This side effect results in substantial discomfort to patients being treated with capecitabine and leads to interruption of treatment. We established the following recommendations for the management of hand-foot syndrome: If grade 2 or 3 hand-and-foot syndrome occurs, it is recommended that the administration of capecitabine be suspended until the event resolves or decreases in intensity to grade 1. Moreover, following grade 3 hand-and-foot syndrome, it is recommended that subsequent doses of capecitabine be decreased. See Package Insert under Dosage and Administration. Interruption of treatment and/or lowering of dose, however, may not be optimal therapy for treating a patient's cancer to achieve the goal and most effectively reduce the tumor burden.

It has now been discovered that capecitabine is especially effective, and best tolerated, in cancer therapy when administered in the specific doses and pursuant to the specific protocols herein described.

A previous experiment entitled “Optimal dosing schedule of capecitabine administration in human mammary xeonograft models” (Experiment 1) has been published. (see ref. Yanigasawa M, Ouchi K, Tanaka Y; Am. Ass. Cancer Res. #3086, 2004). The objective of experiment 1 was to compare three new dosing schedules of capecitabine with the clinical standard schedule (14 days on and 7 days off) at different dose levels and determine treatment efficacy and overall survival.

In experiment 1 female, BALB/c nu/nu athymic nude mice at 4-6 weeks of age were used. Animals had free access to food and water and were housed in a 12-hour light/dark cycle. Per treatment schedule 10 mice were treated per dose level. The experiment consisted of 5 dose levels which contained a control group 4] Capecitabine was administered p.o. at the doses adjusted with total dosage over 6 weeks in each schedule. The total dosages were 22.6; 15.1; 10.1; 6.8 respectively and 0 as control in g/kg/6 weeks. In table 1 the daily dosages per dose level and schedule (+control=0 g/kg/day) are listed and they add up to the same total dosage administered over a total treatment period of 6 weeks.

TABLE 1

| | DI4 | DI3 | DI2 | DI1 |
|---|---|---|---|---|
| Schedule A | 0.808 | 0.539 | 0.359 | 0.241 |
| 14 d on, 7 d off | | | | |
| Schedule B | 0.754 | 0.503 | 0.334 | 0.223 |
| 5 d on, 2 d off | | | | |
| Schedule C | 1.885 | 1.257 | 0.836 | 0.560 |
| 2 d on, 5 d off | | | | |
| Schedule D | 1.077 | 0.718 | 0.477 | 0.320 |
| 7 d on, 7 d off | | | | |

2 human breast cancer xenograft models were used. MX-1 and MAXF 401.Cells were implanted into the mammary fat pad at the right flank of mice at 1.5×10⁶ cells/mouse. Tumors were allowed to establish for 28 days. Mice bearing established tumors were assigned into 16 treatment groups consisting of 10 mice per group as follows: Tumors were measured and ranked according to size and mice bearing excessively small and large tumors were removed from consideration. The remaining mice were distributed into groups with an equal number of mice of each tumor size in each group. Tumor sizes were monitored 3 times per week by caliper measurements for three weeks. Tumor diameters were measured in two orthogonal directions. Tumor volumes were calculated using the following formula:
Tumor Volume(mm³)=Dxd²/2,
where D is the larger diameter and d is the smaller diameter.

All mice were observed and weighed as groups, 7 times per week for three weeks. The average weight of individual mice was calculated by dividing the group weight by the number of animals per group. Percent body weight change was calculated using the formula:
(Current Average Weight−Initial Average Weight/Initial Average Weight)×100.

Measurement of statistical significance of mean tumor volumes between treatment groups was performed using a Wilcoxon Rank Sum Test (p<0.05). Results were reported as tumor growth inhibition after 6 weeks of treatment. For MX-1 and MAXF 401: The antitumor efficacy at non toxic doses in each schedule was equivalent. Schedule C seems to be weaker in toxicity as observed. One explanation is that the daily single dose is very high and most of the mice died even at the lowest daily dosages for this particular schedule in this experimental setting.

Similar anti-tumor activity was observed in all schedules up to 15.1 g/kg/6 weeks (DI3). The toxicities observed particularly in schedule C were more effective than in other schedules due to the higher single daily dose administered. This experiment was designed to compare different schedules of capecitabine at 4 dose levels (plus control) adjusted to the same total dosages over 6 weeks.

### SUMMARY OF THE INVENTION

The present invention relates to a method of treating a patient suffering with cancer, in particular colon or breast cancer, comprising administering to the patient capecitabine in an amount of from about 500 mg/m²/day to about 6000 mg/m²/day, preferably from about 500 to about 3000 mg/m²/day for an administration period of up to about 10 days, preferably up to about 7 days, followed by a rest period of up to about 11 days, preferably 7 days, said administration period starting on the first day of a three week (14 days) to four week (28 day) treatment cycle. Those dosages can be either applied as a body surface area adapted dose (mg/m²/day) or following flat dosing (mg/day). Clinical trials will determine the value of an induction chemotherapy with the highest tolerable dosages used (aiming at 2000 to 6000 mg/m²/day for about 7-10 treatment days with necessary treatment interruption (7-11 days).

### DETAILED DESCRIPTION OF THE INVENTION

As used herein the term “anti-neoplastic” means inhibiting or preventing the development, maturation or proliferation of malignant cells.

The term “therapeutically effective” means an amount of drug, or combination or composition, which is effective for producing a desired therapeutic effect upon administration to a patient, for example, to stem the growth, or result in the shrinkage, of a cancerous tumor.

“Therapeutic index” is a well-recognized term of art and is an important parameter in the selection of anticancer agents for clinical trial. Therapeutic Index takes into consideration the efficacy, pharmacokinetics, metabolism and bioavailability of anticancer agents. See, e.g., J. Natl. Cancer Inst. 81(13): 988-94 (Jul. 5, 1989).

“Tumor control” means that the perpendicular diameters of measurable lesions have not increased by 25% or more from the last measurement. See, e.g., World Health Organization (“WHO”) Handbook for Reporting Results of Cancer Treatment, Geneva (1979).

“Tumor volume (in cubic millimeter)” for purposes of measuring tumor size is calculated using the ellipsoid formula:
(D×(d²))/2
where “D” represents the large diameter of the tumor, and “d” represents the small diameter.

In an embodiment, the present invention relates to a method of treating a patient suffering with cancer, in particular colon or breast cancer, comprising administering to the patient capecitabine in an amount from about 500 mg/m²/day to about 6,000 mg/m²/day, preferably from about 500 mg/m²/day to about 3000 mg/m²/day for up to about 10 days, preferably up to about 7 days, followed by a rest period of up to about 11 days, preferably up to about 7 days, starting on the first day of a two week (14 days) to four week (28 days) treatment cycle, said treatment cycle being repeated every 14-28 days for as long as the tumor remains under control and the regimen is clinically tolerated.

Those dosages can be either applied as a body surface area adapted dose (mg/m²/day) or following flat dosing. Clinical trials will determine the value of an induction chemotherapy with the highest tolerable dosages used (aiming at 2000 to 6000 mg/m²/day for about 7-10 treatment days with necessary treatment interruption (7-11 days). Furthermore the value of a maintenance treatment with the daily dosages in the lower dose range and flat dosing as a continuous daily administration up until progression of the disease will be determined in clinical trials.

A patient's body measurement in square meters (“m²”), this is a “BSA (body surface area”) measurement”, typically ranges from about 1.4 m² to about 2.2 m². Thus, the total amount of capecitabine to be delivered in a treatment cycle (mg) is calculated as follows:
[Dose intensity(mg/m²/week)]×[BSA(m²)]×[number of weeks in treatment cycle]

In a preferred embodiment, capecitabine is administered daily for 7 days, commencing on the first day of a treatment cycle, followed by a rest period of 7 days. The course of a preferred cycle is about 14 to 28 days, though cycles anywhere between about 14 and about 28 days are equally effective and contemplated.

Capecitabine is administered daily preferably divided into two doses, preferably twice per day, most preferably at 12 hour intervals (“Q12” or “BID”). The length of preferred treatment cycle is from about 3 to about 4 weeks.

Capecitabine is administered to the patient in an oral unit dosage form, most preferably in tablet form.

Preferably, the 7 day treatment schedule is repeated every fourteen days, or as soon as permitted by recovery from toxicity, for so long as the tumor is under control or regressing and the patient tolerates the regimen. Preferably, these treatment cycles are repeated for a total of up to about 12 cycles (24 weeks).

In a preferred embodiment the capecitabine is administered twice daily, at a dose of about 700 mg/Q12 (500 mg×1.4 m²) to about 6,600 (3,000×2.2 m²) mg/Q12. In another preferred embodiment, capecitabine is administered twice daily in an amount of from about 750 mg/m²/Q12 to about 3,000 mg/m²/Q12, preferably from about 1,000 mg/m²/Q12 to about to 2,500 mg/m²/Q 12, most preferably from about 1,000 mg/m²/Q12 to about 1500 mg/m²/Q12, for 7 consecutive days commencing on day 1 of a 14 day cycle, followed by a rest period of 7 days. This treatment is repeated every 14 days, or as soon as permitted by recovery from toxicity, for so long as the tumor is under control or regressing and the patient tolerates the regimen. Preferably, the cycles are repeated for a total of up to 12 cycles (that is 24 weeks).

In another preferred embodiment the capecitabine is administered twice daily at a dose of from about 500 mg/m²/Q12 to about 3000 mg/m²/Q12, preferably from about 750 mg/m²/Q12 to about 1500 mg/m²/Q12, for about 5 consecutive days commencing on day 1 of a 28 day cycle, followed by a rest period of 2 days. The cycles will be repeated weekly on day 8(=1).

The determination of tumor control or shrinkage (also referred to as “regression”) is made by known methods. For example, by evaluation of patient symptoms, physical examination, X-ray, MRI or CAT scan or other commonly accepted evaluation modalities.

The present invention may be exemplified by controlled preclinical animal studies as shown in the Example below, which illustrates the invention without limitation.

### Experiment 2

Since Experiment 1 did not provide a dosage regimen having the optimum efficacy/toxicity benefit a new experiment was initiated to explore different schedules at different dose levels but with the same daily dosages, so that a comparison of the different schedules would be possible. Following is the design of the final experiment with the schedules and dosages used (Table 2):

TABLE 2

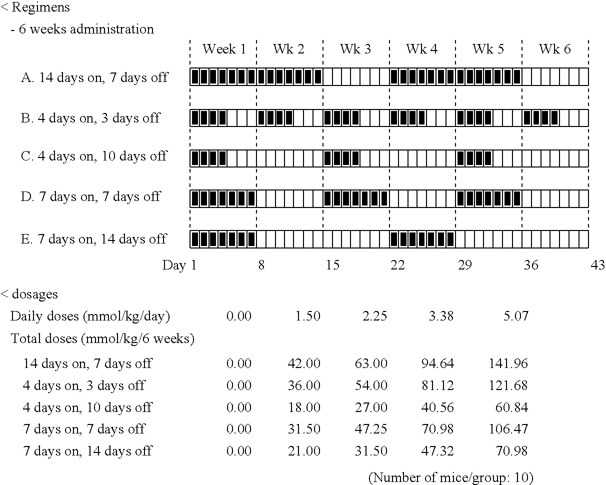

Table 3 demonstrates the survival curve of the treated animals for each treatment schedule. Table 3 reveals that especially in the lower 2 dose ranges the survival curves seem similar for the schedules 14 days on/7 days off and 7 days on/7 days off.

[image]

In Tables 4-1 and 4-2 the tumor volume and body weight changes in a dose comparison for each treatment schedule were noted. Again, in looking at the two lowest dose levels with acceptable side effects, it was observed that a similar tumor volume reduction occurred for the schedules 14 days on/7 days off and 7 days on/7 days off. Within the higher 2 dose levels it was observed that increased levels of toxicity during the 14 days of treatments occurred compared to treatment schedules using shorter treatment durations such as 4 days on/10 days off and 7 days on and 14 days off.

[image]

[image]

In table 5-1 and table 5-2 there is illustrated a schedule comparison at the same daily dosages reflected in changes of tumor volume and body weight per dose level for each treatment schedule. In looking at the lowest dose level with acceptable side effects, there was observed an advantage in efficacy towards the 14 days on/7 days off schedule. However, at higher dose levels 24 there was observed increased levels of toxicity during the 14 days of treatments compared to treatment schedules using shorter treatment durations such as 4 days on/10 days off and 7 days on and 14 days off. Those treatment schedules with a fewer number of treatment days result in higher efficacy at higher dose levels.

[image]

[image]

Summary tables 2-5: The results of the second and final in vivo experiment confirmed that with the same or a better safety profile a higher daily dosage of capecitabine can be applied if you reduce the number of treatment days and then after a rest period introduce capecitabine again. There is evidence that this procedure results in similar or better efficacy (Reduction of tumor volume).

However, the data presented do not finally prove what is happening during the course of a treatment. More specifically, it was necessary to determine at which point in time within 14 days of treatment with capecitabine is only toxicity added but no therapeutic effect anymore documented in the reduction of tumor volume following the daily dosing of capecitabine.

Therefore, the data were then analyzed for the approved (14/7) chemotherapy drug schedule by measuring at each time point after the initiation of therapy the ratio of the (data-derived) expected Gompertzian growth rate in the unperturbed (control) state compared with that observed in the treated state. Curve-fitting was performed by non-linear mixed-effect population modeling using NONMEM software.

The Analysis performed used a non-linear mixed effects population modelling approach with the NONMEM software:

A) with the Gompertzian growth model (TV: tumor volume):
dTV/dt=α*TV−((α/log(TVinf))*TV*log(TV))

B) and the drug effect (DE) model
dTV/dt=α*TV−((α/log(TVinf)*TV*log(TV))*(1−DE)

Results of pooled data with F(x)=DE, F′(x)=rate of change in drug effect (DE) at 1.5 mmol/kg/day and 2.25. mmol/kg/day are demonstrated in Table 6 and Table 7 respectively.

[image]

[image]

Summary of results in Tables 6 and 7:
For 1.5 mmol/kg/day, the drug effect (DE) is maximal, i.e., F′(x)≈0 on Day 27, 10 days after start of treatment (day 17).
For 2.25 mmol/kg/day, the drug effect (DE) is maximal, i.e., F′(x)≈0 on Day 25, 8 days after start of treatment (day 17).

The analysis was also performed on individual data and the drug effect (DE) was estimated for each animal and F′(x) determined.
For 1.5 mmol/kg/day, on average the DE is maximal 8.3 days after start of treatment (range 6-11 days).
And for 2.25 mmol/kg/day, on average the DE is maximal 10.1 days after start of treatment (range 5-18 days).

The time point of maximum impact of treatment is when the absolute value of the ratio of growth rates (perturbed/control) is greatest as determined by methods of calculus. For all dose levels analyzed this point averaged from 8.3-10.1 days into therapy, with the impact of treatment decreasing thereafter despite administration of capecitabine for 14 days. Schedules shorter than 14 days in length can deliver higher dose levels safely. Hence administering one week of treatment followed by 7 days off treatment (7 days on/7 days off) as often as clinically feasible (dose density) provide optimal clinical benefit (efficacy and toxicity).

What is claimed:

1. A method of treating a patient suffering form cancer, comprising administering to said patient a pharmaceutical composition containing as an active ingredient capecitabine in an amount from about 500 mg/m²/day to about 6600 mg/m²/day for up to about 7 days, followed by a rest period of up to about 7 days, said administration starting on the first day of a 14-28 day treatment cycle said treatment cycle being repeated every 14-28 days.

2. A method of treating a patient suffering form cancer, comprising administering to said patient a pharmaceutical composition containing as an active ingredient capecitabine in an amount from about 500 mg/day to about 6600 mg/day, twice daily, for up to about 7 days, followed by a rest period of up to about 7 days, said administration starting on the first day of a 14-28 day treatment cycle, said treatment cycle being repeated every 14-28 days.

3. A method of treating a patient suffering from cancer, comprising administering to said patient a pharmaceutical composition containing as an active ingredient capecitabine in an amount from about 1000 mg/m²/day to about 2500 mg/m²/day for 7 days, followed by a rest period of 7 days, said administration starting on the first day of a 14-28 day treatment cycle, said treatment cycle being repeated every 14-28 days.

4. A method of treating a patient suffering form cancer, comprising administering to said patient a pharmaceutical composition containing as an active ingredient capecitabine in an amount from about 1000 mg/day to about 2500 mg/day, twice daily, for 7 days, followed by a rest period of 7 days, said administration starting on the first day of a 14-28 day treatment cycle, said treatment cycle being repeated every 14-28 days.

5. A method of treating a patient suffering from cancer, comprising administering to said patient a pharmaceutical composition containing as an active ingredient capecitabine in an amount from about 500 mg/m²/day to about 3000 mg/m²/day for 5 days, followed by a rest period of 2 days, said administration starting on the first day of a 7-28 day treatment cycle, said treatment cycle being repeated every 7-28 days.

6. A method of treating a patient suffering from cancer, comprising administering to said patient a pharmaceutical composition containing as an active ingredient capecitabine in an amount from about 500 mg/day to about 3000 mg/day, twice daily, for 5 days, followed by a rest period of 2 days, said administration starting on the first day of a 7-28 day treatment cycle, said treatment cycle being repeated every 7-28 days.

7. The method of claim 1 wherein the cancer is colorectal cancer.

8. The method of claim 1 wherein the cancer is prostate cancer.

9. The method of claim 1 wherein the cancer is lung cancer.

10. The method of claim 1 wherein the cancer is kidney cancer.
